(19) United States
(12) Patent Application Publication
(10) Pub. No.: US 2004/0076630 A1
(43) Pub. Date: Apr. 22, 2004

(54) Method for creating antigens using Emus

(76) Inventors: Maurine Pearson, Flower Mound, TX (US); Teresa Leigh Barr, Port Townsend, WA (US)

Correspondence Address: Wendy KB Buskop, Buskop Law Group, P.C., Suite 500, 1717 St. James Place, Houston, TX, 77056, US

(21) Appl. No.: 10/245,940

(22) Filed: Sep. 18, 2002

(51) Int. Cl. A61K 39/395; C12P 21/04; C07K 16/40

(52) U.S. Cl. 424/146.1; 435/70.21; 530/388.26

(57) ABSTRACT

The invention is a method for creating a vaccine, an anti-venom for snake bites, and an anti-venom for spider bites in an adult ratite bird by obtaining at least one poisonous snake, harvesting venom of the snakes, obtaining an adult ratite bird, injecting the adult ratite bird with the venom in appropriate amounts at appropriate intervals, extracting blood from the bird or harvesting an egg from the inoculated ratite, removing desired antibodies, and using desired antibodies and make an antiserum containing antibodies to the venom.

### FIELD OF THE INVENTION

The present invention relates to compositions and method for use for the treatment of viral infections, venomous snakebites, and poisonous spider bites.

More specifically the present invention relates to compositions and method of use of Emu blood and Emu eggs to create anti-venom serums and viral vaccines.

### BACKGROUND OF THE INVENTION

Found in the wild only in Australia, Emus are the second largest members of the ratite group of flightless birds in the world. The Emu have wings but they are very tiny. They can run up to 30 miles an hour, as they have very large and strong legs. Although a very docile creature, the Emu's legs are so strong; one kick can break a man's leg. Now Emus are being farmed in many parts of the world. They are raised for their valuable products, which include very low fat meat, supple leather hides, decorative and nutritional eggs, and very rich oil, which are obtained from the Emu. Emus are by nature, very healthy and immune to many diseases as well as resistant to parasites such as ticks, fleas, and mites. This immunity makes these birds a perfect choice for growing antigens. Emus are referred to as “living dinosaurs,” as their skeletal structure closely resembles some dinosaurs and in fact, Emus living today closely resemble their ancestors of millions of years ago.

Emu oil is obtained from the fat of the Emu. It is an all-natural substance. Emu oil contains high amounts of EFA's (essential fatty acids). EFA's produce energy in the process of oxidation. In humans EFA's govern growth, vitality and mental state of mind. Oxidation is the central and most important living process in our body. In fact, the EFA structure of the Emu oil is so close to the structure of the human skin oil, it is easy to see from the table below, that Emu products may be less allergenic than other products.

Emus are a flightless bird part of a group called ratites that also includes the ostrich, rhea, cassowary and the kiwi and are indigenous to Africa, South America, Borneo, Australia and New Zealand.

Wherein the fatty acid composition of the Emu oil can be compared to human skin as follows:

| | | | Human |
|---|---|---|---|
| Component | | Emu Oil | Skin Oil |
| Myristic | C:14:0 | 0.3% | 2.1% |
| Palmitic | C:16:0 | 20.3% | 20.2% |
| Palmitoleic | C:16:1 | 3.2% | 3.8% |
| Margaric | C:17:0 | 0.2% | |
| Margaric oleic | C:17:1 | 0.1%% | |
| Stearic | C:18:0 | 10.1% | 11.2% |
| Oleic | C:18:1 | 51.6% | 30.8% |
| Linoleic | C:18:2 | 13.1% | 15.1% |
| Linolenic | C:18:3 | 0.5% | 0.3% |
| Arachidic | C:20:0 | 0.1% | |
| Eicosinoac | C:20:1 | 0.5% | |

| | |
|---|---|
| Calculated iodine value | 69.7-72.8 mEq/100 g |

OSI - 11.95 Hours @ 110.0 degrees

Other fatty acids may be included such as: elaidic and vaccenic.

An analysis of fatty acids in Emu oil reveals that the oil contains approximately 60-70% of fatty acid most of which are unsaturated fatty acid. The major fatty acid found in Emu oil is oleic acid, which is monosaturated and which comprises over 40% of the total fatty acid content of the Emu oil.

Emu oil also contains two essential fatty acids, (EFS's) which are important to human health. 20% linoleic, and 1-2% alpha-linolenic acid.

Essential fatty acids (EFA's) play two important roles in human physiology. Both derive from their incorporation into the phospholipids of cell membranes. By virtue of their high degree of unsaturation, and hence low melting points, they decrease membrane viscosity and affect several aspects of membrane function, including, but not limited to anti-inflammatory properties. An allergenic response is an over-blown inflammation response. The Emu oil reduces inflammation in humans and enhances healing, and therefore the Emu antigens are likely to be less allergenic than that of antigens grown in traditional antigen mediums.

Emus have been tested by Dr. Warren Burggren with the University of North Texas system and have been determined to have a cardiovascular system closely resembling that of humans. Dr. Burggren is quoted to say that “hearts in the eggs of Emus are very similar to human hearts in their early stages of development, page 20, dated 2001, Resource, a periodical published by University of North Texas, Office of University Communications and Marketing published at 3500 Camp Bowie Blvd, Ft. Worth Tex., 76107-2699.

In looking at the profile of the similarities between human skin oil, as well as lipid profile and the similarity between the human heart and the Emu, it would be likely a common thread exists between the two. As well as the fact that emus are disease resistant and have a low immunology profile, antigens grown in emus create less allergenic reaction than antigens grown in traditional mediums.

A need has long existed for a method for making vaccines and anti-venom serums that do not require the use of horses, chicken eggs, sheep, porcine (pig) pancreatic hydrolysate of casein, VERO cells; a continuous line of monkey kidney cells, or fetal rhesus, monkey lung cells, as well as yeast derivatives.

Emu oil is more compatible with the human body oil, and produces fewer allergic reactions. Many,people cannot receive life saving vaccines because they are allergic to chicken or horse and their bodies cannot tolerate the presence of those components in the serum. A need has existed for vaccines, particularly for small pox, diphtheria, measles, mumps, rubella, polio, tetanus, pertusis (whooping cough), HIB, pneumonia, meningitis, influenza, hepatitis A, tuberculosis, and cholera that were not derived from chicken eggs.

An invention that could multiply snake venom would be beneficial to humans. An invention that could utilize using the Emu similar composition and heart embryos could reduce the risk of allergic reactions in humans. The composition of Emu oil and skin oil are close in comparison, as is the hearts of Emus and humans. Dr. Warren Burggren has also discovered the same common thread and similarity between the cardiovascular systems of snake embryos and human heart embryos. This would further the argument for using the Emu, as at one level, the Emu, the snake and the human all have a “common thread” according to Burggren. The present invention would be more compatible to human beings than using horse serum or chicken eggs, therefore decreasing allergic reaction to vaccines and anti-venoms.

Snakes also propagate very badly and slowly in captivity, so to obtain their venom they often have to be caught in the wild; another lengthy, difficult and dangerous task. Besides this, the venom composition depends on the snake's age, sex, season and other factors. For all of these reasons, the ideal snake anti-venom production system would be one that didn't involve the reptiles at all to multiply the anti-venom. Emus are a natural choice to multiply anti-venom, as they are disease resistant an their cardiovascular systems as well as skin oil is similar humans.

Emus are from the ratite family. The ratite family includes the Emu, ostrich, rhea, cassowary, and kiwi. Emu eggs have a volume 500-850 ml and are equivalent to about 10-15 large chicken eggs. Without the shell, chicken eggs contain about 65% white, and 35% yolk. By comparison, Emu eggs contain 55% white and 44% yolk. This is one noticeable difference between chicken and Emu eggs. The Emu's incubation period is 56 days as versus the chicken at 21 days. The chicken egg contains approximately 43.0% cholesterol compared to 89.0% in Emu eggs. Emu's eggs also contain less water than chicken eggs, 67.0% compared to 75.0% in chicken eggs. The white of the Emu eggs contains mostly water, about 90%, with about 9% protein and no cholesterol. The yolk of the Emu contains 45-50.0% moisture, 15% protein, 1.5% cholesterol and 30-40.0% lipids. The high protein content of the white of the Emu egg make it a perfect choice for growing antigens for antibiotic production and vaccines, as antigens attach themselves to the protein of the embryonic fluid. Emu eggs would be preferable as their considerable size and potential for vaccine production. An Emu egg is approximately 10 to 15 times larger with a higher egg white percentage as well as protein content.

Vaccines grown in Emu serum are less likely not to cause allergic reactions such as tetanus shots, in humans due to the closeness of compatibility.

An invention is needed that could increase the production of antigens for antibiotics, vaccines and the like 10 to 15 times faster than chicken eggs would be desirable and save lives. An invention that could grow more vaccine at one time for harvest would be beneficial to humans and animals.

### SUMMARY

The invention is a method for creating anti-venom for snakebites in an adult ratite bird. The method involves obtaining a poisonous snake, harvesting the venom, obtaining an adult ratite bird, injecting the bird with the venom or other antigen in small amounts, and increasing the tolerance of the ratite bird to the venom. The venom injections are continued until the bird does not exhibit symptoms of the venom in the bird. The method then involves extracting blood from the bird, removing desired antibodies, and using the desired antibodies to make an antiserum containing antibodies to the venom.

The invention is also a method for creating anti-venom for spider bites in an adult ratite bird. The method is similar to the snakebites, except that the venom of a spider is used in an adult ratite bird. The method then involves extracting blood from the bird, removing desired antibodies, and using the desired antibodies to make an antiserum containing antibodies to the venom.

The invention is also a method for making a vaccine using the egg of a ratite bird by obtaining an embryonated egg of a ratite bird, obtaining seed viruses in a suitable carrier, inoculating the egg with the seed virus using a syringe, and, then, incubating the egg. The method involves separating the albumin from the yoke of the egg, removing the protein from the antibodies forming an antibody concentrate, and mixing the antibody concentrate with a carrier.

The invention is also a vaccine for one of the following of any existing live and killed pandemic viruses such as but not limited to influenza virus, mumps virus, measles virus, rubella virus, diphtheria virus, tetanus virus, small pox virus, tuberculosis, rotavirus, varicella, pertusis (whooping cough), HIB, pneumoccal, meningoccal, cholera, rabies virus and poliovirus.

### DETAILED DESCRIPTION OF THE PREFERRED EMBODIMENTS

A method for creating a serum for combating snake bites, the flu, any pandemic virus, and similar viruses using ratite birds, in particular the Emu, to create an anti-snakebite serum or vaccine which is less allergic than similar viruses or anti-venoms grown in horses, chicken eggs, sheep, porcine (pig) pancreatic hydrolysate of casein, VERO cells; a continuous line of monley kidney cells, or fetal rhesus, monkey lung cells, as well as yeast derivatives.

The method uses Emu eggs that are ten to fifteen times larger than chicken eggs to grow vaccines for various types of virus, including small pox, any pandemic virus and others. The resultant vaccine causes fewer allergic reactions in users and is more compatible with human blood systems than known vaccines and anti-snake bite serums grown in chicken eggs. The method of the invention is much less expensive than known chicken egg methods, because it takes fifteen chicken eggs and fifteen chickens to create the same quantity of vaccine as in one Emu egg.

Since it takes approximately fifteen chickens a day to lay fifteen eggs, this inventive method saves the filth of fifteen chickens, the food for fifteen chickens, the space of fifteen chickens, the need for chicken coops, the need for chicken medicines to keep the chickens healthy. In contrast, an Emu can lay nine eggs per month, the equivalent 135 chicken eggs per month. Emus are not as fussy as chickens. Unlike chickens, Emu's are weather tolerant and can get wet, stay outside in the cold under freezing temperatures (as long as they don't get frostbitten) and be outside in the open up 110° F. shade or no-shade. Emus eat bugs, grasses, and have no need for special food in order to lay eggs, although a Emu supplemental feed is favorable to ensure optimum health and overall wellness of the birds.

Overall, since fifteen chickens cost more to maintain and have a higher chance of dying than one Emus, and since the average Emu lives to be about 30 years old and the average chicken only lives to be about 7 years old, this inventive method is more economical by almost 50% than known techniques.

Further, a typical Emu costs, at most about $65 per year to feed and maintain.

A typical chicken costs about $20-25 per year. Clearly, this method using Emus and ratite birds is much less expensive than conventional methods using chicken eggs to grow vaccines.

A large number of viruses can be used to create vaccines by this method, including but not limited to influenza virus, mumps virus, measles virus, rubella virus, diphtheria virus, tetanus virus, small pox virus, tuberculosis pertusis (whooping cough), HIB, rotavirus, varicella, pneumoccal, meningoccal, cholera, rabies virus and poliovirus.

The Emu embryos have a cardiovascular system that is similar to human cardiovascular systems. The Emu oil is similar to human skin oil. Since they are similar, the anti-biotics and vaccines are better grown in Emu eggs versus chicken eggs because they can hold more volume and make produce more antibody. This is especially necessary since influenza vaccines are grown for up to 4 months. Using larger eggs, such as from the ratite family, specifically Emu eggs would be beneficial. The present invention is based upon the theory that Emu egg embryos cardiovascular system are close to humans, can house more vaccine, are therefore more compatible and reduce allergic reactions to vaccinations and the like. The problem with growing vaccines using animal cells that are not human like is that during serial passage of the virus thru the animal cells, is that animal RNA and DNA can be transferred from one host to another and undetected animal viruses may slip past quality control testing procedures as in 1955 thru 1961 with SV40 which stands for simian virus #40, meaning the 40th virus found which has oncogenic or cancer causing properties.

The invention is a method for creating anti-venom for snakebites in a ratite bird. The method involves obtaining at least one poisonous snake, harvesting venom of the snakes, obtaining a ratite bird, inoculating or injecting the adult ratite bird with the venom, serum or virus in amounts in order to induce antibody formation or disease-producing antigens, thereby causing the Emu to create immunity to the disease. An inoculated bird from the ratite family would then lay eggs with ready-made antibodies. In some cases, an automated laser flow-cytometric method may be used to tabulate and validate antigen levels in the ratite bloodstream. Serum can also be extracted from the inoculated ratite, thereby, extracting blood from the bird. The serum is the fluid that separates from clotted blood, similar to plasma but without clotting agents, also called blood serum, The desired antibodies are removed, and using the desired antibodies and make an serum or antitoxin containing one or more specific ready made antibodies used to provide immunity against a disease or counteract venom from snakes and spiders, then making a preparation containing the weakened or dead microbes of that venom that cause the particular disease or discomfort, and administered to stimulate the immune system to produce antibodies against that disease in humans and animals. Antivenom acts to neutralize the poisonous venom of snakes and spiders and causes the venom to be released from the receptor site. Thus, the receptor sites that were previously blocked by venom are now free to interact with the acetylcholine molecule, and normal respiration resumes in the victim. The spent antivenom and the neutralized venom are then excreted from the body.

The production of anti-venom for snakebites method can further involve extracting one percent of blood of the Emu that can be collected via the jugular vein or wing, using a 3-10 ml syringe and a 20-22 gauge needle. The serum or antitoxin is the fluid that separates from clotted blood, similar to plasma but without clotting agents, also called blood serum.

The micro-organisms, or serum, either bacteria or viruses, thought to be causing certain infectious diseases and which the vaccine is supposed to prevent are whole-cell proteins or just the broken-cell protein envelopes, and are called antigens. Antigens are a substance, usually a protein, on the surface of a cell or bacterium that stimulates the production of an antibody. Chemical substances that are supposed to enhance the immune response to the vaccine, called adjuvants, which is a drug or agent added to another drug or agent to enhance its medical effectiveness and is a substance injected along with an antigen to enhance the immune response stimulated by the antigen. Chemical substances which act as preservatives and tissue fixatives, which are supposed to halt any further chemical reactions and putrefaction such as decomposition or multiplication of the live or attenuated or killed biological constituents of the vaccine.

Adjuvants, preservatives and tissue fixatives can be formaldehyde, thimerosal, aluminum hydroxides and aluminum phosphates, polysorbates 80 and 20, gelatin, hydrolyzed gelatin and processed gelatin, glycerol, sucrose, sorbitol, formalin, sodium chloride, phenoxyethanol, betapropiolactone, phenol red, monosodium glutamate, potassium monophosphates, tri (n) buytlphosphate, lactose, ammonium sulfate, residual MRC5 from the medium, and the like.

The desired antibodies in the method can consist from influenza virus, mumps virus, measles virus, rubella virus, diphtheria virus, tetanus virus, small pox virus, tuberculosis, rotavirus, varicella, pertusis (whooping cough), HIB, pneumoccal, meningoccal, cholera, rabies virus, and poliovirus, snake and spider anti-venom.

The method's anti-serum can comprise suitable adjuvants for injection, such as, adjuvants, tissue fixatives and preservatives, formaldehyde, thimerosal, aluminum hydroxides and aluminum phosphates, polysorbates 80 and 20, gelatin, hydrolyzed gelatin and processed gelatin, glycerol, sucrose, sorbitol, formalin, sodium chloride, phenoxyethanol, betapropiolactone, phenol red, monosodium glutamate, potassium monophosphates, tri (n) buytlphosphate, lactose, ammonium sulfate, residual MRC5 from the medium, and the like.

The injections in the method can be in the amount of 1-3 ml per day.

The ratite bird in the method for creating anti-venom for snakebites can be an Emu.

The invention is also snake anti-venom created by the method for creating anti-venom for snakebites. The invention is also snake anti-venom made by the method for creating anti-venom for snakebites using an Emu.

The snakes used in the method for creating anti-venom for snakebites anti-venom and, therefore, the snake anti-venom can be from the following group: coral snake, pit viper, copper head, cottonmouth, rattle snake, western Mississauga snake, water moccasin, western pigmy snake, western diamond back, cane brake snake, Mojave snake, mottled rock snake, banded rock snake, black tailed snake, prairie snake, and south Texas rattle snake. The list of snakes also pertains to the snake anti-venom created using an Emu.

The invention is a method for creating anti-venom for spider bites in an adult ratite bird. The method involves obtaining at least one poisonous spider, harvesting venom of the spider, obtaining an adult ratite bird, injecting the adult ratite bird with the venom in small amounts, increasing the tolerance of the ratite bird to the venom, continue venom injections daily until the bird does not exhibit symptoms of the venom in the bird, extracting blood from the bird, removing the desired antibodies and using antibodies and make an antiserum containing antibodies to venom.

The method for creating anti-venom for spider bites can include between 6. 0% and 10.0% of the blood volume of the adult ratite every 2 to 3 weeks to be removed. In the method, venom can be injected in amounts appropriate to innoculate the adult ratite bird.

The adult ratite bird in the method for creating anti-venom for spider bites can be an Emu.

The invention is also a spider anti-venom made by the method for creating anti-venom for spider bites. The spider anti-venom can be made by the method for creating anti-venom for spider bites using an Emu.

The spiders used in the method for creating anti-venom for spider bites anti-venom and, therefore, the spider anti-venom can be from the following group: brown recluse spider, black widow spider, brown spider, widow spider, red widow spider, and northern widow spider.

The invention is also a method for making a vaccine using the egg of a ratite bird. The method involves obtaining an adult ratite bird, obtaining seed viruses in a suitable carrier, inoculating the egg adult ratite with the seed virus using a syringe, obtaining an egg from the inoculated bird separating the albumin from the yoke of the egg, removing the protein from the antibodies forming an antibody concentrate, and mixing the antibody concentrate with a carrier, adjuvants, tissue fixatives and preservatives.

The seed viruses used in the method for making a vaccine using the egg of a ratite bird can be a member of the following group: live and killed pandemic viruses, which include, influenza virus, mumps virus, measles virus, rubella virus, diphtheria virus, tetanus virus, small pox virus, rabies virus and polio, tuberculosis pertusis (whooping cough), HIB, rotavirus, varicella, pneumoccal, meningoccal, cholera, rabies virus and poliovirus.

The incubation period in the method for making a vaccine using the egg of a ratite bird can be one week when the seed virus is a stockpiled pandemic virus. The protein in the method can be removed from the albumin by extraction. The carrier in the method can be adjuvants, tissue fixatives and preservatives, formaldehyde, thimerosal, aluminum hydroxides and aluminum phosphates, polysorbates 80 and 20, gelatin, hydrolyzed gelatin and processed gelatin, glycerol, sucrose, sorbitol, formalin, sodium chloride, phenoxyethanol, betapropiolactone, phenol red, monosodium glutamate, potassium monophosphates, tri (n) buytlphosphate, lactose, ammonium sulfate, residual MRC5 from the medium, and the like.

The invention is also a vaccine for a member of the group of live and killed pandemic virus, influenza virus, mumps virus, measles virus, rubella virus, diphtheria virus, tetanus virus, small poxvirus, rabies virus and poliovirus, tuberculosis pertusis (whooping cough), HIB, rotavirus, varicella, pneumoccal, meningoccal, cholera, rabies virus and poliovirus.

While only a few embodiments of the invention have been disclosed in the above detailed description, the invention is not limited thereto but is susceptible to various changes without departing from the scope of the invention.

What is claimed is:

1. A method for creating an anti-venom for snake bites in an adult ratite bird comprising the steps of:
a. obtaining at least one poisonous snake;
b. harvesting venom of the snakes;
c. obtaining an adult ratite bird;
d. injecting the adult ratite bird with the venom in appropriate amounts at appropriate intervals;
e. extracting blood from the bird or harvesting an egg from the inoculated ratite;
f. removing desired antibodies; and
g. using desired antibodies and make an antiserum containing antibodies to the venom.

2. The method of claim 1, wherein 6.0% to 10.0% of the blood of the bird is extracted and removed at 2 to 3 week intervals.

3. The method of claim 1, wherein the desired antibodies are consisting from the group live and killed pandemic viruses, which include, influenza virus, mumps virus, measles virus, rubella virus, diphtheria virus, tetanus virus, small pox virus, rabies virus and polio, tuberculosis pertusis (whooping cough), HIB, rotavirus, varicella, pneumoccal, meningoccal, cholera, rabies virus and poliovirus.

4. The method of claim 1, wherein the antiserum comprises an adjuvant, preservative and tissue fixative chosen from the following group: formaldehyde, thimerosal, aluminum hydroxides and aluminum phosphates, polysorbates 80 and 20, gelatin, hydrolyzed gelatin and processed gelatin, glycerol, sucrose, sorbitol, formalin, sodium chloride, phenoxyethanol, betapropiolactone, phenol red, monosodium glutamate, potassium monophosphates, tri (n) buytlphosphate, lactose, ammonium sulfate, residual MRC5 from the medium, and the like.

5. The method of claim 1, wherein the venom is injected in amounts 0.001 ml to 10 ml per day.

6. The method of claim 1, wherein the adult ratite bird is an Emu.

7. A snake anti-venom made by a method for creating the snake anti-venom in an adult ratite bird comprising the steps of:
a. obtaining at least one poisonous snake;
b. harvesting venom of the snakes;
c. obtaining an adult ratite bird;
d. injecting the adult ratite bird with the venom in appropriate amounts at appropriate intervals;
e. extracting blood from the bird or harvesting an egg from the inoculated ratite;
f. removing desired antibodies; and
g. using desired antibodies and make an antiserum containing antibodies to the venom.

8. A snake anti-venom of claim 7, wherein the adult ratite bird is an Emu.

9. A snake anti-venom of claim 7, wherein the snake is a member of the group: coral snake, pit viper, copper head, cottonmouth, rattle snake, western Mississauga snake, water moccasin, western pigmy snake, western diamond back, cane brake snake, Mojave snake, mottled rock snake, banded rock snake, black tailed snake, prairie snake, and south Texas rattle snake.

10. A method for creating an anti-venom for spider bites in an adult ratite bird comprising the steps of:
a. obtaining at least one poisonous spider;
b. harvesting venom of the spider;
c. obtaining an adult ratite bird;
d. injecting the adult ratite bird with the venom in small amounts;
e. increasing the tolerance of the ratite bird to the venom;
f. continue venom injections daily until the bird does not exhibit symptoms of the venom in the bird;
g. extracting blood from the bird;
h. removing the desired antibodies; and
i. using antibodies and make an antiserum containing antibodies to venom.

11. The method of claim 10, wherein between 6.0% and 10.0% of the Emu blood is removed at 2 to 3 week intervals.

12. The method of claim 10, wherein the venom is injected in amounts 1-3 ml per day.

13. The method of claim 10, wherein the adult ratite bird is an Emu.

14. A spider anti-venom made by a method for creating the spider anti-venom in an adult ratite bird comprising the steps of:
a. obtaining at least one poisonous spider;
b. harvesting venom of the spider;
c. obtaining an adult ratite bird;
d. injecting the adult ratite bird with the venom in small amounts;
e. increasing the tolerance of the ratite bird to the venom;
f. continue venom injections daily until the bird does not exhibit symptoms of the venom in the bird;
g. extracting blood from the bird;
h. removing the desired antibodies; and
i. using antibodies and make an antiserum containing antibodies to venom.

15. The spider anti-venom of claim 15, wherein the adult ratite bird is an Emu.

16. The spider anti-venom of claim 15, wherein the spider is a member of the group: brown recluse spider, black widow spider, brown spider, widow spider, red widow spider, and northern widow spider.

17. A method for making a vaccine using the egg of a ratite bird comprising:
a. obtaining an adult ratite bird;
b. obtaining seed viruses in a suitable carrier;
c. inoculating the ratite with the seed virus using a syringe;
d. obtaining the egg of an inoculated ratite bird;
e. separating the albumin from the yoke of the egg;
f. removing the protein containing antigens forming an antibody concentrate; and
g. mixing the antibody concentrate with a suitable adjuvant, tissue fixative and preservative.

18. The method of claim 17, wherein the seed viruses are selected from a member of the group consisting of live and killed pandemic viruses, influenza virus, mumps virus, measles virus, rubella virus, diphtheria virus, tetanus virus, small pox virus, tuberculosis pertusis (whooping cough), HIB, rotavirus, varicella, pneumoccal, meningoccal, cholera, rabies virus and poliovirus.

19. The method of claim 17, wherein the incubation period is one week when the seed virus is a stockpiled pandemic virus.

20. The method of claim 17, wherein the antigen is removed from the egg by extracting the embryonic fluid and then extracting the protein cells which contain the adhered antigens.

21. The method of claim 17, wherein the carrier is sterilized water.

22. A vaccine for a member of the group of live and killed pandemic viruses, influenza virus, mumps virus, measles virus, rubella virus, diphtheria virus, tetanus virus, small pox virus, tuberculosis pertusis (whooping cough), HIB, rotavirus, varicella, pneumoccal, meningoccal, cholera, rabies virus and poliovirus.
